(19) United States
(12) Patent Application Publication
(10) Pub. No.: US 2008/0255461 A1
(43) Pub. Date: Oct. 16, 2008

(54) Real-time optical monitoring system and method for thermal therapy treatment

(76) Inventors: Robert Weersink, Toronto (CA); Lee Chin, North York (CA); John Trachtenberg, Toronto (CA); Alex Vitkin, Toronto (CA); William Whelan, Charlottetown (CA); Brian C. Wilson, Toronto (CA)

Correspondence Address: Mark A. Litman & Associates, P.A.;York Business Center, Suite 205, 3209 West 76th St., Edina, MN, 55435, US

(21) Appl. No.: 12/079,418

(22) Filed: Mar. 26, 2008

Related U.S. Application Data
(60) Provisional application No. 60/920,091, filed on Mar. 26, 2007.

(51) Int. Cl. A61B 5/00; A61B 18/18

(52) U.S. Cl. 600/476; 606/10

(57) ABSTRACT

Multiple site information of light intensity is obtained by application of a multiple-fibre probe in a real-time optical monitoring system. The multiple-fibre probe includes a plurality of optical fibres distributed along the length of the probe. Each optical fibre may be is switchable between the mode for transmitting optical signal into the malignant tissue and the mode for collecting the optical signal from the same tissue. Thus the numbers of the probes can be minimized for collecting multiple site light information and the irritation to the tissue is reduced. A method of using such a probe to determine coagulated boundary in thermal or other treatment is also described.

### RELATED APPLICATION DATA

This application claims priority from U.S. Provisional Patent Application Ser. No. 60/920,091 filed Mar. 26, 2007.

### BACKGROUND OF THE INVENTION

1. Field of the Invention

The invention relates to an optical monitoring system and method for detecting the boundary(s) of coagulated tissue, and more specifically, to a real-time optical monitoring system and method for differentiating coagulated tissue from normal tissue and determining boundary(s) of at least one coagulated zone that may be used in thermal or like treatment of malignant tissue, such as cancer tissue and especially prostate cancer tissue, to ensure its complete treatment and protection of valuable normal tissue.

2. Background of the Art

Interstitial thermal therapies (ITT) are minimally invasive treatment modalities that employ high temperatures (50-90° C.) to achieve destruction of cancerous tumours and benign lesions. Thin needle applicators, fiber bundles, or catheters are inserted into the tumour site and energized with lasers, microwaves, radio frequency (rf or RF), or ultrasound. The energy is absorbed over a volume determined by the energy penetration properties. Heating the tumour site results in coagulative necrosis. Due to the complex and dynamic nature of tissue properties of the tumour tissue during ITT, the development of real-time monitoring systems that can accurately detect tissue damage during the procedure and preserve critical normal structures is vital. Temperature sensors are commonly used to monitor and control ITT treatments. However, due to slow thermal conduction to, into or through the sensors, the localized nature of the measurement, patient-specific variability in thermal damage, and the inability to directly sense tissue coagulation, temperature measurements alone might be insufficient for assessing treatment efficacy or even avoiding significant damage to benign tissue.

Recent research has demonstrated that optical point monitoring, where light intensity information is measured using interstitial optical sensors, provides near instantaneous response to structural changes that occur during treatment. This is due to the fact that the scattering coefficient of most mammalian tissue increases significantly (between 2 and 7 times) upon thermal coagulation. The sharp difference in scattering between coagulated and native tissue enables optical monitoring of the changes in the tissue with increasing coagulation, especially in the procedure when the applicators inserted in the tissue are energized with laser light, known as laser interstitial thermal therapy (LITT), or interstitial laser photocoagulation (ILP), interstitial laser thermotherapy (ILT), or laser-induced thermal therapy (also LITT).

Optical monitoring employs the use of interstitially placed fiber based optical sensors to measure changes in detected optical signal during LITT to monitor the location of the boundary of thermally damaged (coagulated) tissue, based on the significant change in optical properties of tissue due to coagulation. The change in optical attenuation can, therefore, be detected if an interstitially implanted light source and detector(s) are used to survey the targeted tissue, to ensure the complete treatment of the malignant tissue and protection of valuable normal tissue.

The laser source employed to ‘probe’ the target tissue may originate either from the treatment fibre employed to heat the tissue (i.e., for LITT) or from another implanted laser fibre delivering laser energy at low laser powers that result in biologically insignificant increases in temperature (i.e., diagnostic source). In the latter case, the technology may also be employed for monitoring of non-laser interstitial thermal therapies (i.e., microwave, radiofrequency and ultrasound) as the optical properties of coagulated tissues change regardless of heating modality.

The utility of optical non-directional light intensity (i.e., fluence) sensors for monitoring the interstitial therapies has been reported in Whelan W M; Chun P; Chin L C L; Sherar M D and Vitkin I A, “Laser thermal therapy: utility of interstitial fluence monitoring for locating optical sensors”, Phys. Med. Biol. 46 N91-N96 (hereinafter “Reference 1”); Chin L C L; Whelan W M; Sherar M D and Vitkin I A, “Change in relative light fluence measured during laser heating: implications for optical monitoring and modelling of interstitial laser photocoagulation”, Phys. Med. Biol. 46 2407-2420 (hereinafter “Reference 2”); and Chin L C L; Whelan W M; and Vitkin I A, “Models and measurements of light intensity changes during laser interstitial thermal therapy: Implications for optical monitoring of the coagulation boundary location”, Phys. Med. Biol. 2003 February 48(4):543-59 (hereinafter “Reference 3”). In 2003, the use of directional light intensity (i.e., radiance) resulted in significant improvements in sensitivity for detecting the coagulated damage boundary, as reported in Chin L C L; Pop M; Whelan W M; Sherar M D and Vitkin I A, “An optical method using fluence or radiance measurements to monitor thermal therapy”, Rev. Sci. Intrum. 74(1) 393-395 (hereinafter “Reference 4”); and Chin L C L; Wilson B C; Whelan W M; and Vitkin I A, “Radiance-based monitoring of the extent of tissue coagulation during laser interstitial thermal therapy”, Opt Lett. 29(9):959-61 (hereinafter “Reference 5”).

These various references demonstrate the development in the field. For example, Reference 1 presented a strategy for determining the position of an array of optical sensors; Chin L C; Whelan W M; and Vitkin I A, “Scattering weight functions for interpreting dynamic fluence changes during LITT Phys Med Biol” (in press) (hereinafter “Reference 8”) and Reference 2 disclosed that interstitial fluence signals may be employed for the detection of tissue charring and the onset and growth of the thermal damage boundary; Reference 2 and L. C. L. Chin, SR Davidson; W. M. Whelan; M. D. Sherar and I. A. Vitkin, “Optical monitoring of interstitial laser photocoagulation,” Physics in Canada, Vol. 59, p. 99, 2003 (hereinafter “reference 14”) disclosed employing interstitial fluence signals combined with diffuse light models for assessing the location of the coagulation boundary prior to passing of the optical sensor location; Reference 3 disclosed employing interstitial fluence signals for the detection of the passing of the thermal damage boundary at the sensor location; Reference 4, Reference 5 and Lee CL Chin; William M Whelan and I Alex Vitkin, “A novel optical feedback strategy for monitoring interstitial laser photocoagulation”, Lasers in Medical Sciences, Vol. 18 (Suppl. 1), p. S41, 2003 (hereinafter “Reference 17”) employed interstitial radiance signals for detecting the onset and approach (sensor pointing at source) and passing (sensor point away from source) of the thermal damage boundary at the sensor location; Reference 4, Reference 5 and L C L Chin; W M Whelan; SR Davidson and I A Vitkin, “Interstitial optical-based reconstruction of thermal coagulation during microwave thermal therapy”, Physics in Canada, Vol. 60, p. 61, 2004 (hereinafter “Reference 15”) disclosed employing interstitial optical signals (fluence) for the monitoring of non-laser interstitial thermal therapies; References 1-5 used an optical fibre (fluence or radiance tip) coupled to a photodiode or photomultiplier tube as a sensor; References 1 and 3 employed multiple sensors at different positions although different probes are or may be required for each position and that each probe houses only a single sensing fibre; In Reference 5 radiance detection was performed using a single rotating sensor to acquire directional information at different detection angles; References 1-5 used only a single wavelength for monitoring purposes; Reference 14 disclosed that there is a potential to evaluate the specific absorption rate in the target tissue in vivo; Reference 15 utilized interstitial light intensity data across multiple projections in the tissue to reconstruct 2D maps of thermal coagulation; W. M. Whelan; L. C. L. Chin; M. M. Brookshaw and I. A. Vitkin, “Interstitial optical measurements: A new approach to guiding laser therapies” Physics in Canada, Vol. 61, 2005 (hereinafter “Reference 16”) used radiance measurements coupled with a P3 approximation optical model recover unique optical properties of a turbid medium; Reference 17 disclosed that optical readings are indicative of a larger sampling volume than temperature readings.

Conventionally, radiance measurements occur at only a single point for each probe, while measurements are taken continuously throughout the thermal treatment. A single point measurement, however, only provides feedback from one location in the tissue, whereas the therapy is targeting a much larger volume of tissue. A single point measurement may also be prone to errors in assessing the change in coagulation since it may be influenced by specific local factors in the tissue, such as the presence of a large artery or vein.

Systems that both provide radiation through sets of optical fibers and retrieve affected radiation through geometrically disposed receiving fibers are known in gross measurement anatomical situations such as shown in U.S. Pat. No. 7,142,307 (Stark). The Stark patent discloses apparatus and methods for the simultaneous or rapid sequential use of two or more different separations between the source and detector of the measuring apparatus to obtain spectral measurement data in diffuse transmission or “interaction” modes of collecting optical information from a specimen. The method and apparatus subsequently combine separate data taken from two or more different pathlengths to provide discrimination against undesired information while preserving or enhancing desired information. Additional reference information to normalize the optical signal is also provided. The optical and mechanical design of the optical probe also provides for transmittance, reflectance and interactance measurements on small amounts of specimen. The system does not provide for real time field viewing, but detection of differences along different path lengths.

U.S. Pat. No. 4,884,891 (Borsboom) describes a fibre-optic apparatus for determining in a material a phenomenon affected by light back-scattered by surface and/or volume refraction. The apparatus comprises a light source; an illuminating system connecting the light source to a sensor head; a light detection system connected to the sensor head; and within the sensor head an optical illuminating fibre connected to the illuminating system, and a juxtaposed optical detection fibre connected to the light detection system. The optical fibres are mounted in a mutually fixed position. In the sensor head according to the invention at least one solid optical illuminating fibre and at least one juxtaposed optical detection fibre are disposed with their optical axes parallel to each other through an axial length from the end of the optical fibres arranged to face the material to be examined, and the optical illuminating fibre is adapted to be also used as an optical detection fibre in addition to the optical detection fibre first mentioned. The system is used in imaging technology for image evaluation.

### SUMMARY OF THE INVENTION

Employing several sensing positions in a real-time optical monitoring system can provide feedback from multiple positions in the target volume and overcome the disadvantage of single point measurement. The information collected from each sensor can be used to create a real-time map of coagulated damage.

According to one aspect of the invention, multiple optical probes collect optical information from overlapping areas or volumes of tissue during LITT to provide optical data on scattering effects by tissue.

Thus the monitoring system is able to monitor changes in tissue properties due to coagulation at several sites within the tissue with minimum irritation. Based on these changes, the changing boundary between the normal and coagulated tissue during a thermal coagulation therapy can be ‘mapped’ or located in a 3D image.

### BRIEF DESCRIPTION OF THE DRAWINGS

The invention will be better understood and will become apparent when consideration is given to the following detailed description thereof. Such description makes reference to the annexed drawings wherein:

FIG. 1 is a schematic diagram of a real-time optical monitoring system according to one embodiment of the invention;

FIG. 1A is a schematic diagram of a real-time optical monitoring system according to one embodiment of the invention;

FIGS. 2A and 2B is are respectively the side view and end view of part of a probe in FIG. 1 in an enlarged view;

FIG. 3 illustrates a measuring configuration when using a monitoring system according to one embodiment;

FIG. 4 illustrates the measured signal change in an ex vivo tissue sample treated using interstitial laser thermal therapy detected according to the configuration in FIG. 3;

FIG. 5 illustrates the calculated signal change in a similar tissue sample as treated in FIG. 4, detected according to the configuration in FIG. 3;

FIGS. 6A and 6B illustrate another measuring configuration according to another embodiment.

### DETAILED DESCRIPTION OF THE INVENTION

The technology described herein relates to methods, apparatus and systems for assisting in the performance of invasive therapeutic procedures on tissue and especially tissue subjected to coagulation treatments, especially cancerous tissue generally and cancerous tissue of the prostate gland to remove malignant tissue. An optical monitoring system differentiates coagulated tissue (densified tissue, coagulation implying blood clotting within the environment, in medical procedures a potentially intentional procedure to cause cell death by coagulation) from non-coagulated tissue in an organ in tissue treatment. The system may basically have numerous components, such as at least one probe comprising a housing and at least two optical elements housed within the probe and distributed along the length of the probe. Each of the optical elements may be switchable or shiftable between at least two different functional capacities or modes. One mode includes emitting visible (optical) or detectable (e.g., mechanically detectable such as ultraviolet radiation, near infrared radiation or far infrared radiation) radiation. The radiation may be transmitted through the optical element (e.g., optical fiber transmission, light piping, etc.) or originate within an element (semiconductor, diode, etc.) and output at, on or into the tissue. The probe may have at least a second optical element and collecting at least some of the optical output affected by the tissue. The term “affected by the tissue” means that the light is partially reflected, refracted, dispersed, or otherwise altered in a measurable fashion, especially in a fashion that alters with coagulation of the tissue. The emission or provision of detectable radiation and its interactance with the tissue (on the surface or from within the tissue to projection out of the surface of the tissue) provides detectable and/or measurable changes in the radiation which are suggestive of, probative of or definitive of coagulation or other proposed change in the tissue from the therapeutic treatment. The system may also have a control (manual or automated) for configuring, directing, controlling or effecting whether and when each or some of said elements a) transmit or emit optical signal (continuous beam, pulse, waves, etc.) into or onto the target tissue, e.g., malignant tissue or b) collect the optical signal from the same tissue. The control may enable one of the at least two optical elements to emit optical output and another of the at least two optical elements to collect the at least some optical output affected by the tissue. The affecting by the tissue is typically on an optical property selected from the group consisting of optical density, reflectivity, radiance and light dispersion.

Instead of a set of switchable optical devices, the optical monitoring system for differentiating coagulated tissue from non-coagulated tissue in an organ in tissue treatment may have a) at least one probe comprising a housing, and b) at least two optical elements housed within the probe and distributed along the length of the probe. The at least two optical elements would have at least one radiation emitting optical element and at least one other optical element that was an optical receiver for collecting at least some of the optical output affected by the tissue. The collection may be converted to meaningful data locally, converted to electronic signals of light intensity locally, or the light may be transmitted to a more distal element where the light is read and converted into optical data. There should be at least one display system in communication with optical receiver (either directly or more likely through a processor or data storage system so that meaningful and quality image data can be provided) for displaying image data or images based upon collected optical output.

The system may also have a light source; a detector for detecting the optical output affected by the tissue collected (the output being collected or detected by the collecting optical elements; each optical element may have a first end proximal to the tissue and a second end distal from target tissue. The control may have a first set of terminals connected to a light source and the detectors, and a second set(s) of terminals that are connected to the second ends of said optical elements. The control can switch modes (individually or in tandem or sequence) at least two optical elements to effect transmittal of the optical signal from the light source to the tissue or to the detector for collecting the at least some optical output affected by the tissue. The system may further have an analyzer (e.g., comprising a processor) for analyzing and interpreting signals detected by the detector, and possibly an electronic visual display for displaying result of analysis or printout. A light source may be present in the system and the light source is selected from coherent and incoherent light sources, as those sources are well understood in the art. The analyzer may contain software or an algorithm which inserts the data taken from the probe and evaluates, compares or measures absolute and/or relative values of the light to assemble an image or relative analysis of tissue within the region being viewed by the probe.

It is preferred that the housing fully enclose the at least two optical elements so that environmental liquids do not contact the optical elements, which could cause deteroriation in materials, irregularly block or mask light emission and/or collection, or otherwise interfere with the process. The exterior of the housing is either hydrophilic (to allow local liquids to evenly coat the outside surface of the housing to reduce bubbles or waves or spots forming on the surface) or highly hydrophobic to avoid attraction or adherence of liquid to the surface. The housing may also comprise two separate enclosing components (two separate but adjacent closed pipette or tube) that may be fixed with respect to distance from each other. For example, one tube may carry light emitters, and another parallel tube may carry the collectors, with both tubes supported on a frame or yoke. In this way, some defined space is created between the two optical elements to assure activity on the emitted radiation by tissue. Because the spacing, shape and geometry of the separation between the two elements and the shape of tissue between the two elements can be defined by the separation, consistent measurements may be provided. For example, if each tube had a pointed pyramidal shape, the ends could be pressed against tissue and the shape of tissue conforming to the space between the pyramidal tips would always be of a consistent shape (pyramidal itself). This would provide accurate readings.

The light source may be a continuous single wave light source, an amplitude modulated light source or a pulsed light source. The optical elements may comprise optical fibres. A micro-prism may be mounted on the tip of each optical fibre to selectively accept optical signals and/or each optical fibre may be an angle-cleaved or bevelled fibre. The system is preferably used in the treatment of malignancies, such as where the target tissue is prostate tissue. Each of said optical fibres may be so configured that the optical signal emitted or collected approximates perpendicularity to the length of the probe and in a known particular direction. Amplitude modulation is preferably used for 2 reasons. One can measure tissue properties by using amplitude modulated light at very high frequencies and looking at the change in phase and/or modulation, rather than only the amplitude of the signal. This can also be done using pulsed light and fast detectors. The other, and more common reason, for using amplitude modulated light is because it can improve signal to noise ratios, particularly if one also uses lock-in amplifiers, or phase detection. This system would only measure the amplitude of signals at a particular frequency. This is a common technique in signal processing, and is usually performed at much lower frequencies than 5 MHz. Although with frequency ranges greater than 5 MHz the operation without phase detection is simplified, making a system using lock-in detection enables use of a wider, and particularly lower range of frequencies, such as 0.01 MHz, 0.05 MHz, 1.0 MHz and higher.

At least some optical elements may comprise light emitting diodes for a light source and photodiode detectors for light detection.

An optical monitoring method using this technology may used for differentiating coagulated tissue from non-coagulated tissue in an organ in target tissue during therapeutic treatment (e.g., thermal treatment provided by a thermal source) that alters the physical and especially optical properties of the tissue. The method may include:
applying at least one probe on at least one side of the thermal source, the at least one probe comprising the system described herein, such as with a housing, and at least two optical elements housed within the at least one probe and distributed along the length of the at least one probe. Each of the at least two optical elements may be switchable between emitting of an optical signal at target tissue and collecting an optical signal returned from the target tissue. The method includes emitting or transmitting a source light via an emitting optical element at the target tissue; collecting optical signal by the collecting optical elements of the probe; treating target tissue (e.g., in a manner that alters or is intended to alter the optical properties of the target tissue); and measuring signal change of the collected optical signal from treated target tissue. The treatment of the target tissue preferably effects coagulation of the target tissue. The method may further include determining an extent of coagulation by interpreting the measured signal change.

LITT may use optical energy delivered via thin, flexible fibres or bundles of fibers that are typically inserted directly into tissue to thermally destroy solid tumours. In regions heated to greater than about 55° C., irreversible coagulative cell death occurs, which is manifested immediately and grows outward from the source fibre(s) as treatment progresses. The primary goal of LITT is the complete thermal destruction of the target tumour while sparing the surrounding healthy tissue. It is desirable and often necessary to monitor LITT to provide real-time feed-back information regarding the size, spatial extent, and location of the damage volume throughout the course of this dynamic treatment.

As a result of coagulation, the optical properties of tissue in the coagulated zone change significantly. For example, the scattering coefficient of coagulated tissue increases significantly and results in a dynamically changing light distribution that can be detected and measured in real time by use of interstitial optical sensors, such as optical fibres. Fluence or radiance of light can be measured for the change of the optical characteristics to determine the coagulation boundary. If fibres are used as sensors, the measurement of fluence or radiance may lie in the different optical fibre tip configuration, the use of multiple sensors in a single probe and any associated actuation mechanism used to position or rotate the fibres/sensors in the probe housing, since the collection fibres may need to be moved linearly to different positions (e.g., pulling back on it) in order for the all-direction light measurement, or the radiance fibres may need to be rotated angularly to enable different viewing directions at the same tip location.

As mentioned above, though conventional radiance measurements occur at only a single point for each probe, it has some disadvantage in the measurement. Employing several sensing positions provides feedback from multiple positions in the target volume, and the information collected from each sensor can be used to create a map of coagulated damage. Because inserting multiple probes into the tissue is irritating, by employing multiple sensors in one probe, it reduces the irritation for the tissue.

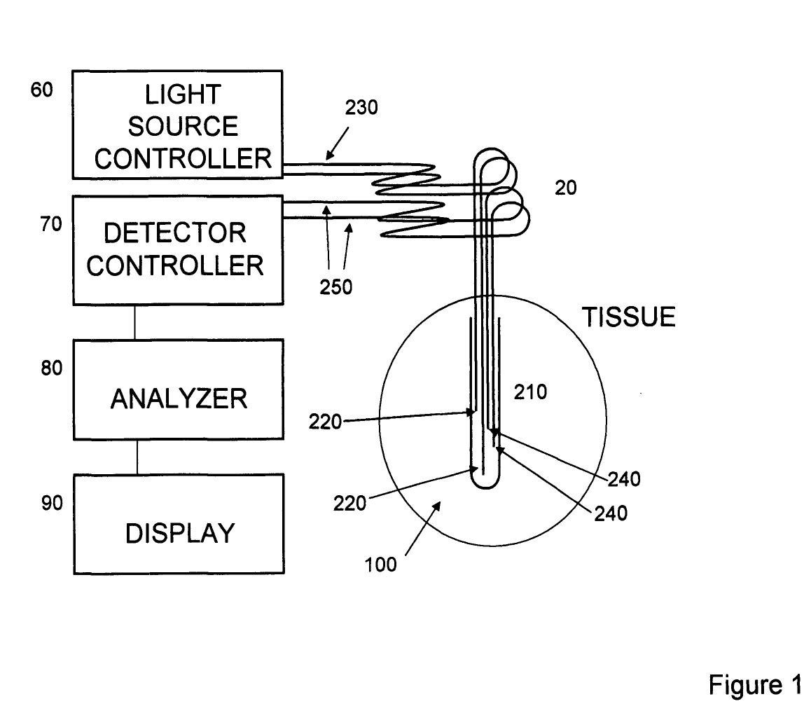

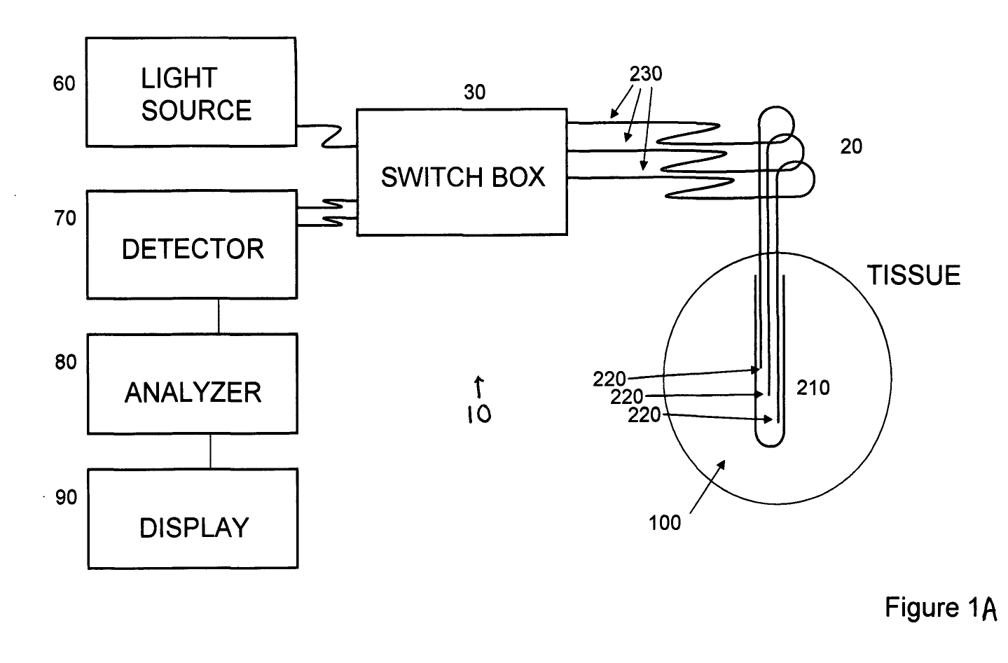

FIG. 1 is a schematic diagram of a real-time optical monitoring system according to one embodiment of the invention. The real-time monitoring system 10 includes a probe 20, a light source controller 60, a detector controller 70, both of which are connected to the probe 20, an analyzer 80 and a display 90.

FIG. 1A is a schematic diagram of a real-time optical monitoring system 10 according to one embodiment of the invention, with a switch box 30 intermediate elements in the system 10.

The probe includes a sheath 210 (shown in part 1), a plurality of sources 220 and a plurality of detectors 240 housed in the sheath 210. The light sources 220 are connected to the light source controller by a cable 230. The detectors 240 are connected to the detector controller by a cable 250. Light from the source is directed into the tissue 100 where it is scattered and attenuated by the tissue prior to being detected. This will be referred to as the optical signal. Though FIG. 1 only shows one probe and one optical fibre switch box, multiple probes and switch boxes can be used in the system, as shown in FIG. 1A.

FIG. 1 is a schematic diagram of a real-time optical monitoring system according to one embodiment of the invention. The real-time monitoring system 10 includes a probe 20, a light source 60, a detector 70, an optional optic fibre switch box 30 (in FIG. 1A) one end of which is connected to the probe 20 and the other end to the light source 60 and detector 70, an analyzer 80 and a display 90.

The probe includes a sheath 210 (shown in part 1) and a plurality of optical fibres 240 housed in the sheath 210. Each fibre 240 has a distal end 220 adjacent to or into tissue 100, and a proximal end 230 away from the tissue 100. Depending on the connection on the proximal end 230, each optical fibre 240 can work as either a transmitter or as a receiver. When the switch box 30 connects a fibre 240 to the light source 60, the fibre 240 will act as a transmitter and deliver source light from the light source 60 into the tissue 100, via switch box 30 (in FIG. 1A). When the switch box 30 connects a fibre 240 to the detector 70, the fibre 240 will act as a receiver and collect light from the source that has been scattered and attenuated by the tissue. This will be referred to as the optical signal or scattered light in the tissue 100 to the detector 70 via switch box 30. Though FIG. 1A only shows one probe and one optical fibre switch box, multiple probes and switch boxes can be used in the system.

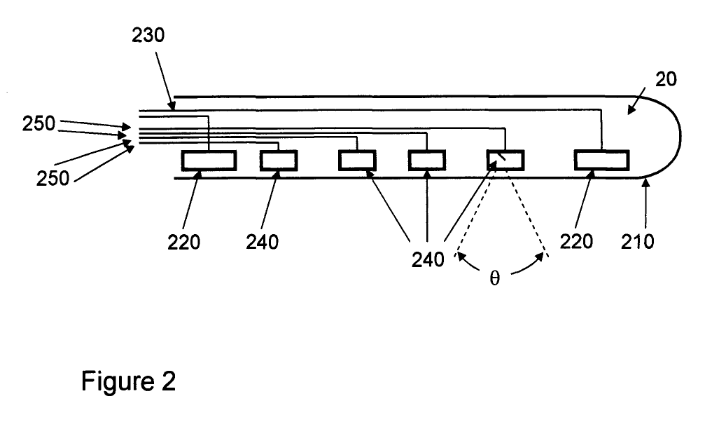

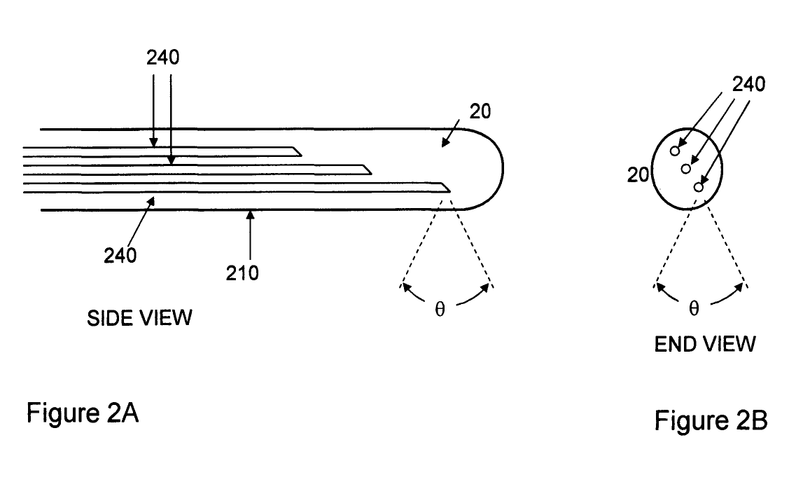

FIG. 2 is the side view of the distal end of a probe in a larger scale. The plurality of sources 230 and plurality of detectors 240 are distributed along the length of the probe 20, preferably equally spaced from one another so that the optical signals at several different sites may be measured. In one embodiment, radiance is measured and a photodiode detector may be used to detect only photons from a certain direction perpendicular to the length of the probe 20 and into a solid angle θ as shown in FIG. 2. The range of the solid angle θ is in the range of 0-180°. More detailed description of the solid angle θ is set out below.

FIGS. 2A and 2B are, respectively, the side view and end view of the distal end of a probe in a larger scale. The plurality of fibres 240 are distributed along the length of the probe 20, preferably equally spaced from one another so that the optical signals at several different sites may be measured. In one embodiment, radiance is measured and a micro-prism may be used at the fibre tip or an angle-cleaved fibre may be used to accept only photons from a certain direction and with a certain acceptance angle about that direction, in such a manner that the light is reflected perpendicular to the length of the probe 20 and into a small solid angle θ as shown in both FIGS. 2A and 2B. The range of the solid angle θ is in the range of 0-180°. More detailed description of the solid angle θ is set out below.

Referring back to FIG. 1A, the fibre optic switch box 30 is used to change the connection between each optical fibre between either the source or detection mode, in another word, between transmitter and receiver mode. The switch box 30 has terminals to connect to the proximal ends of the optical fibres 240 in the probe 20, and terminals to connect to the light source 60 and the terminals to the detector 70. Depending on the settings, each fibre 40 may be switchably connected by the switch box 30 to either the light source 60 or the detector 70, but not to both at the same time. For a better result, one fibre may be connected to the light source 60 working as a transmitter, and all the rest fibres to the detector 70 working as receivers. By switching the connections of the fibres 240 to either the light source 60 or the detector 70, the position of source light can be changed without physically changing the position of the probe 20, thus minimizing the number of probes inserted into the tissue and the need to move the probes, and thereby reducing irritation to the subject. The switch box 30 available in the market, such as the Intelligent Optical Switch from Glimmer (Hayward, Calif.) can be used in the system. The light source 60 is typically a laser source, but it may be a light emitting diode or a lamp preferably with light in the near infrared region of the spectrum i.e., 600-1000 nm. The source light can be either continuous (i.e. no change in intensity over time), frequency modulated, or amplitude modulated or pulsed.

The detector 70 will transform the optical signal to a form suitable for measurement and include but not limited to photoelectric sensor or the like. The collecting or sensing optical element may be a sensor that transmits an electronic signal as well as an optical fiber or a diode to emit radiation. The switchable technique may be as simple as activating (powering) one electronic component (emitting versus sensing) or the other.

The analyzer 80 will analyze the detected signal using the well-known models of light propagation in tissue, such as Monte Carlo (MC) or Finite Element calculations or the like. In order not to dilute the invention, further details of the analysis of these signals will be not described. The result of the analysis can be shown in the display 90, which may be a LCD indicator or a monitor or screen for displaying a map derived from the analysis.

When light is traveling in tissue, it is generally scattered in all directions. As described above, fluence rate measurement would measure the intensity of light coming from all directions. Radiance measurement would only measure the light coming from one direction. But we can define this one direction to be light coming from one direction, with a tolerance (or acceptable limits) defined by a solid angle of directions around this one direction. That is, a solid angle is effectively the volume of a cone, the exterior of which cone is defined by rotation of a line about a central point (the head of the cone).

Using radiance measurements, the fibre can be oriented so that the solid collection angle is either facing the thermal source, or away from the thermal source. The light signal collected during a thermal therapy will be different depending on these orientations. In order to avoid the difference of the result caused by whether the fibre is facing the thermal source or away from it, measurements at more than one orientation can also be conducted and combined into one set of data

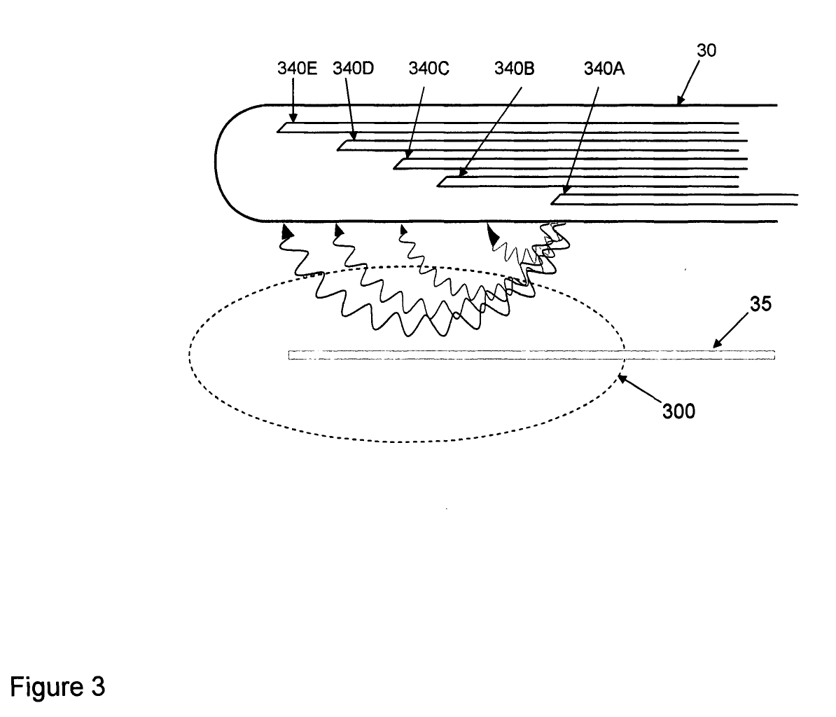

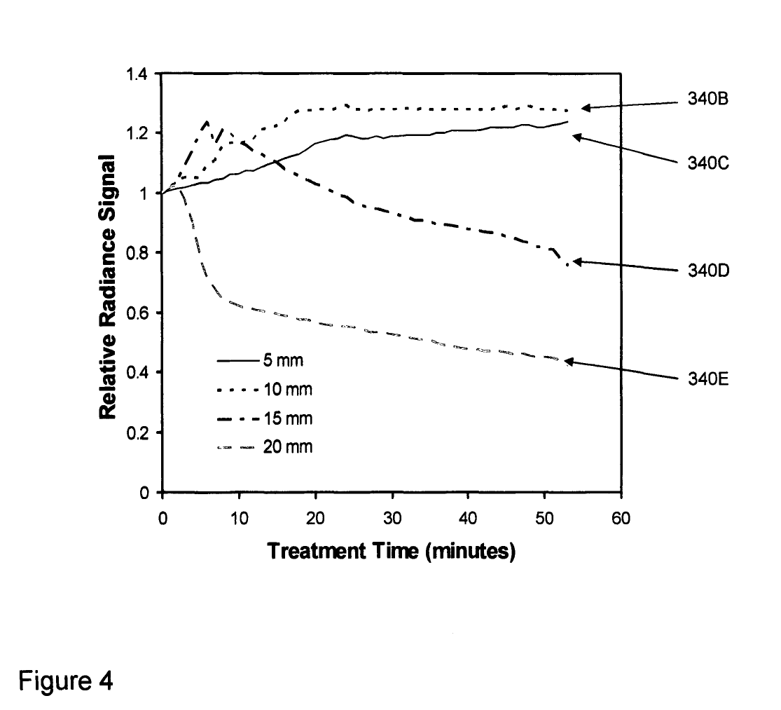

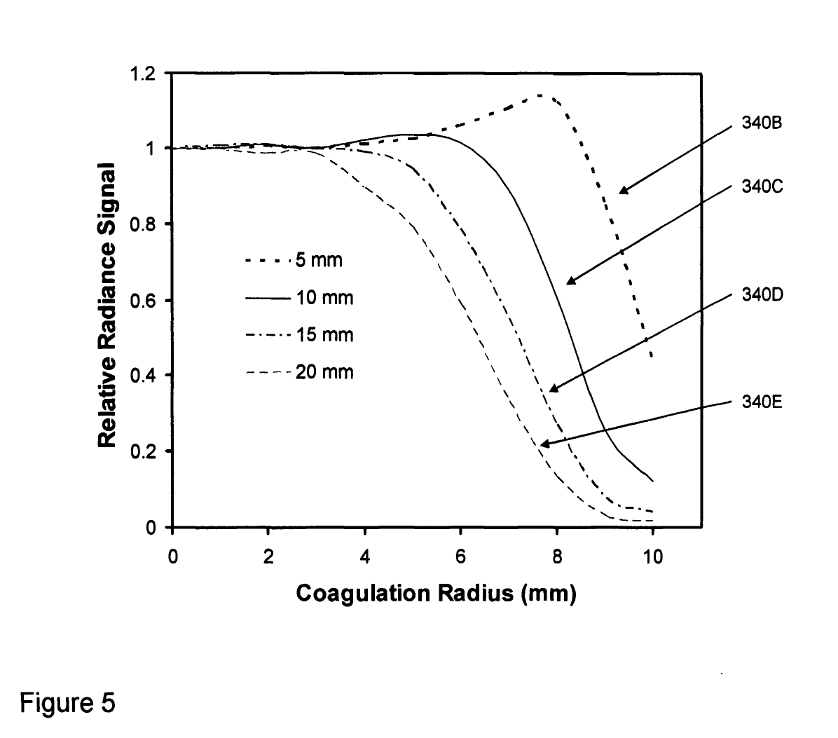

FIG. 3 illustrates another possible embodiment using a monitoring system where only one five-fibre probe is used. The probe 30 has five optical fibres and fibre 340A works as a transmitter and fibres 340B, 340C, 340D and 340E as receivers. The probe 30 is located on one side of the thermal source 35 in the suspected malignant tissue and there is only one transmitter used in this embodiment. The light transmits from the fibre 340A into the tissue. With the increasing coagulated extent, the radiance signal (as well the fluence signal) will decrease. The fibres closer to the transmitter 340A will see a change in radiance later than those fibres farther from the transmitter in the probe. Light collected at 340B travels only a short distance towards the heat source, while light collected at 340E travels closer to the heat source. Hence, light collected at 340E will “see” the coagulated front sooner that light collected at 340B. FIG. 4 illustrates the radiance change detected by fibres 340B, 340C, 340D and 340E along with the treatment time during a laser thermal treatment of an ex vivo tissue sample of bovine muscle. Details of the measurement are given below. FIG. 5 illustrates the results of Monte Carlo calculations of the radiance detected by fibres 340B, 340C, 340D and 340E along with the radius of coagulation zone 300 as shown in FIG. 3 surrounding the laser thermal treatment of an ex vivo tissue sample of bovine muscle. Details of the calculations and are given below. Analysis of these signals will not be described in more detail, as it embodies well understood analytic procedures in which specific properties of the collected radiation are associated with the expected changes in the optical properties and physical/chemical properties of the target issue (e.g., coagulation). Preferred analysis will provide the extent of coagulated zone 300 as shown in the figure.

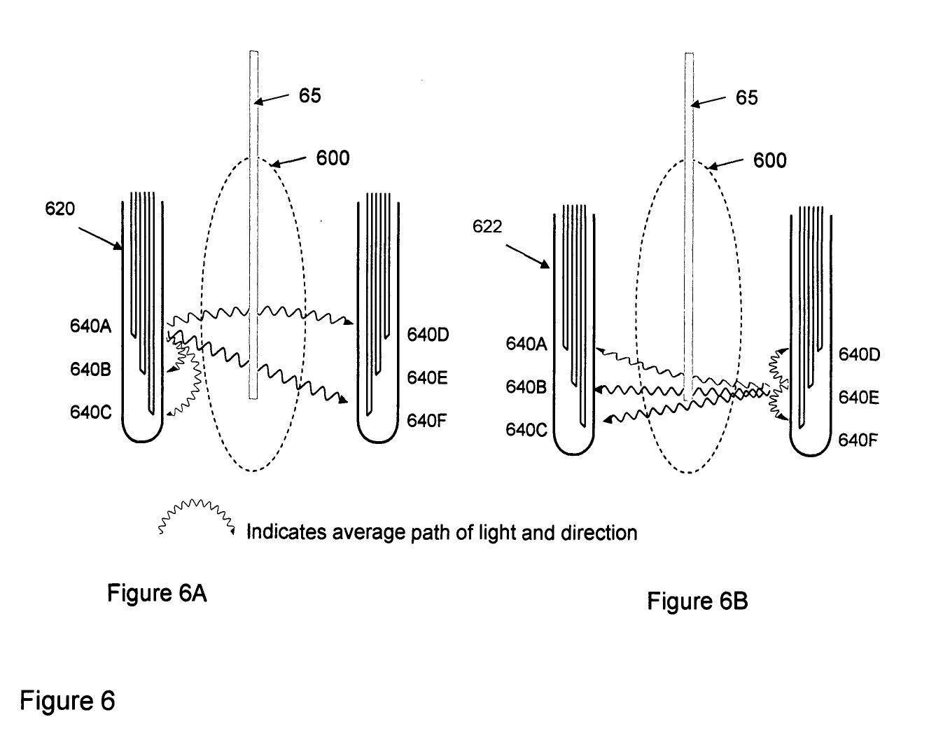

FIGS. 6A and 6B illustrate another measurement configuration when using a monitoring system according to another embodiment, in which two three-fibre probes 620 and 622 are used. The two probes 620 and 622 are inserted on the opposite side of the thermal source 65 in the anticipated coagulated tissue so that the thermal source 65 is between the probes. The volume of the coagulated tissue will increase radially from this thermal source 65 in the process of thermal treatment. In FIG. 6A, the optical fibre 640A acts as a transmitter and fibres 640B and 640C in the probe 620 and all the probes 640D, 640E and 640F in the probe 622 act as receivers and collect radiance data. As shown in the figure, light travelling between 640A and 640D will “probe” the volume of tissue between these two fibres. Light travelling between 640A and 640C will “probe”, or travel through, a different volume of tissue. The changes in the radiance signal measured at 640D will therefore be indicative of the extent or expansion of the coagulated tissue in the region close to fibre 640D. Likewise, the radiance signal at 640C will be indicative of the extent or expansion of the coagulated tissue in the region close to fibre 540C. Based on the changing radiance signals collected from each fibre, and hence the extent or expansion of the coagulated tissue at the position of each sensor, the position of the boundary 6500 between the coagulated and untreated tissue can be defined in the plane defined by the two probes 620 and 622 and the thermal source 65.

Changing the position of the transmitter (for example from 640A to 640E) will enable greater sampling of the tissue, and improve the accuracy of the position of the boundary between the coagulated and normal tissue. By operating the switch box 30 and switching on individual light source in a probe, the source light position can be easily changed without pulling out the probes and re-inserting them back into the tissue. FIG. 6B shows the geometry after the transmitter position is changed to fibre 640E. A second set of signals reflecting the radiance difference can be obtained by operating the switch box 30, without moving or reinserting any probe.

Again, analysis of these signals will provide the extent of coagulated zone and a general “map” of the extent of tissue coagulation can be derived. The more sets of signals are obtained, the more accurate the result of the analysis will be. Thus this map would provide significantly more information to the user regarding the extent of damage with only a small increase in the number of probes that need to be inserted into the tissue. The resolution of this map is a function of the number of sources and sensors, and the number of probes inserted into the tissue. The greater the number of fibres in one probe and/or the greater the number of the probes, in other words, the greater the number of sensors, the better the resolution of the map will be. In the above example, the mapping is across a single plane, however, it is not limited in a single plane and inserting probes in other planes provides information in three dimensions.

In the above embodiment, light radiance at multiple sites are measured and analyzed. Please note that light fluence can also be measured for the analysis. In that case, a small scattering or diffusing tip may be added to the tip of the distal end of each fibre for collecting the overall direction-independent light intensity, namely, fluence. The rest of the system may remain the same. Though optical fibres are described in the embodiment, it is appreciated that the invention is not limited to optical fibres only. Other optical elements such as photodiode detectors or any light sensing device can be used for measurement of light fluence or radiance too. It is also appreciated that, although the figures only show 3 or 4 sensors, the number of sensors can normally vary from 2 to 5, and may be even higher depending on the volume of tissue to be monitored and the dimensions of the probe housing the sensors. It is important to note that the analysis does not depend on the absolute value of light intensity, namely fluence and/or radiance, but depends on the intensity change along the time and/or the relative intensity values among different receivers.

Though optical fibres are described as an example for collecting and sensing the light transmitted in the tissue throughout the application, other small light sensors can also be used for the probe, such as photodiode detectors, but not limited to these, and can be arranged for measuring either fluence or radiance. Though the description uses “optical signal,” “radiation” and “light”, it is well known that they have the same meaning.

We now describe in detail an example of measurements using the embodiment described in FIG. 3. Measurements were made on ex vivo bovine muscle. Tissue coagulation was produced using a laser coupled to a 10 mm diffusing tip fiber. Radiance probes were made by inserting optical fibers into the tissue with the fiber ends facing the thermal source. The fibers were spaced 5 mm apart along a line that was parallel to the thermal source fiber and separated from the thermal source by 10 mm. This is an equivalent geometry to the embodiment shown in FIG. 3. One of the fibers was connected to a low power laser at 760 nm (referred to as the probe light and identical to 340A in FIG. 3) while the other fibers (340B, 340C, 340D and 340E) were connected to photodiode detectors. After collecting baseline optical signals using the probe light, treatment was initiated and optical signals collected every 30 seconds. The changes in optical signals relative to the baseline measurements are shown in FIG. 4. The optical signal collected at 20 mm (340E) quickly drops, while the relative signal at 15 mm (340D) initially increases, then decreases. Optical signals at 10 and 5 mm's (340C and 340B respectively) increase at different rates. Eventually the change in optical signals decreases. At the completion of treatment, the tissue sample was cut open and the lesion diameter was measured to be 6 mm. To analyze the measured optical signals, Monte Carlo calculations were made of the same optical arrangement. Optical properties of normal and coagulated bovine tissue were taken from the literature (Reference 18, Roggan, A; Dorschel, K; Minet, O; Wolff, D; Muller, G, Laser-induced Interstitial Thermotherapy, ed. G. Muller, A. Roggan, SPIE, Bellingham, 1995). A layer of coagulated tissue was centered at 10 mm under the tissue surface. Calculations were made with the thickness of the coagulation layer increasing from 0-10 mm in radius. The probe light source and radiance collection was positioned as in the measurement above and as shown in FIG. 3. The relative changes in optical signals are shown in FIG. 5. The Monte Carlo calculations show that as the coagulation radius increases, the relative optical signal at 20 mm decreases rapidly. The relative optical signal at 5 mm (340B) increases, and eventually decreases when the coagulation radius gets larger than 5 mm.

While not identical, the measurements and calculations show similar trends. The optical signal measured at the 20 mm sensor (340E) decreases soon after the start of treatment, while the signals at the other sensors respond at different times after the start of treatment and generally correspond to increasing of coagulation. At 5 and 10 mm (340B and 340C), the measurements and calculations show an increase in optical signal, followed by a decrease but the increase is smaller in the calculations. Comparing the measured optical signals at the end of the treatment with the calculated optical signals, the predicted coagulation radius is approximately 7 mm, slightly larger than the measured radius of 6 mm.

Although the invention has been described in connection with preferred embodiments, it should be understood that various modifications, additions and alterations may be made to the invention by one skilled in the art without departing from the spirit and scope of the invention as defined in the appended claims.

What is claimed is:

1. An optical monitoring system for differentiating coagulated tissue from non-coagulated tissue in an organ in tissue treatment, the system comprising:
at least one probe comprising a housing, and at least two optical elements housed within the probe and distributed along the length of the probe,
each of said optical elements being switchable between emitting optical output into target tissue and collecting at least some of the optical output affected by the targeted tissue.

2. The system of claim 1, further comprising a control for configuring whether each of said elements for transmitting optical signal into the targeted tissue or for collecting the optical signal from the same targeted tissue.

3. The system of claim 2 wherein the control can enable one of the at least two optical elements to emit optical output and another of the at least two optical elements to collect the at least some optical output affected by the targeted tissue.

4. The system of claim 3 wherein the affecting by the targeted tissue is on an optical property selected from the group consisting of optical density, reflectivity, radiance, fluence and light dispersion.

5. The system of claim 1, further comprising a light source; a detector for detecting the optical output affected by the targeted tissue collected by the collecting optical elements; each said optical element having a first end proximal to the targeted tissue and a second end distal from the targeted tissue, said control having a first set of terminals connected to a light source and the detectors and a second sets of terminals connected to the second ends of said optical elements to switch individually at least two optical elements to effect transmittal of the optical signal from the light source to the targeted tissue or to the detector for collecting the at least some optical output affected by the targeted tissue.

6. The system of claim 5, further comprising an analyzer for analyzing and interpreting signals detected by the detector, and an electronic visual display for displaying result of analysis.

7. The system of claim 2, wherein a light source is present in the system and the light source is selected from coherent and incoherent light sources,

8. The system of claim 2, wherein the light source is a continuous single wave light source.

9. The system of claim 2, wherein the light source is an amplitude modulated light source.

10. The system of claim 2, wherein the light source is a pulsed light source.

11. The system of claim 2, wherein the optical elements comprise optical fibres.

12. The system of claim 11, wherein a micro-prism is mounted on the tip of each optical fibre to selectively accept optical signals.

13. The system of claim 11, wherein each of said optical fibres is an angle-cleaved or bevelled fibre.

14. The system of claim 11, wherein said target tissue is prostate tissue.

15. The system of claim 14, wherein each of said optical fibres is so configured that the optical signal emitted or collected approximates perpendicularity to the length of the probe and in a directional manner encompassing at most a hemisphere.

16. The system of claim 2, at least some optical elements comprise light emitting diodes for a light source and photodiode detectors for light detection

17. An optical monitoring method for differentiating coagulated tissue from non-coagulated tissue in an organ in target tissue during thermal treatment provided by a thermal source, the method comprising:
applying a probe on at least one side of the thermal source, said at least one probe comprising a housing, and at least two optical elements housed within the at least one probe and distributed along the length of the at least one probe,
each of said the at least two optical elements being switchable between emitting of an optical signal at target tissue and collecting an optical signal returned from the target tissue;
emitting a source light via an emitting optical element at the target tissue;
collecting optical signal by the collecting optical elements of the probe;
treating target tissue; and
measuring signal change of the collected optical signal from treated target tissue.

18. The method of claim 17 wherein the treatment of the target tissue effects coagulation of the target tissue.

19. The method of claim 18 further comprising determining an extent of coagulation by interpreting the measured signal change.

20. The method of claim 17, further comprising changing the position of the source light by switching at least one of the optical elements from an emitting mode to a collecting mode.

21. The method of claim 20, wherein the optical elements comprise optical fibres.

22. The method of claim 18, wherein the signal change is radiance change and each fibre is so configured so as to accept only selected directional optical signal.

23. The method of claim 21, wherein each of optical fibre comprises an angle-cleaved fibre or has a micro-prism mounted thereon.

24. The method of claim 21, wherein each of said optical fibres is so configured that the optical signal emitted or collected approximates perpendicularity to a length of the probe.

25. The method of claim 21, wherein the source light is lamp light or laser.

26. The method of claim 21, wherein the source light is a continuous single wave light.

27. The method of claim 21, wherein the light source is a modulated light.

28. The method of claim 21, wherein the light source is a modulated light at a frequency greater than 5 MHz.

29. The method of claim 21, further comprising applying a second probe on an opposite side of the thermal source.

30. The method of claim 29, wherein said at least one probe and the second probe and said thermal source are in the same plane.

31. An optical monitoring system for differentiating coagulated tissue from non-coagulated tissue in an organ in tissue treatment, the system comprising:
at least one probe comprising a housing, and at least two optical elements housed within the probe and distributed along the length of the probe,
the at least two optical elements comprising at least one radiation emitting optical element and at least one other optical element comprising an optical receiver for collecting at least some of the optical output affected by the tissue; and
at least one display system in communication with optical receiver for displaying image data or images based upon collected optical output.
